(19) United States
(12) Patent Application Publication
(10) Pub. No.: US 2025/0075161 A1
(43) Pub. Date: Mar. 6, 2025

(54) Highly-breathable Closed Cell Culture Device and Method

(71) Applicant: SUZHOU CELLPRO BIOTECHNOLOGY CO., LTD., Suzhou (CN)

(72) Inventors: Shengyou ZHANG, Suzhou (CN); Zhuqing YE, Suzhou (CN)

(73) Assignee: SUZHOU CELLPRO BIOTECHNOLOGY CO., LTD., Suzhou (CN)

(21) Appl. No.: 18/748,138

(22) Filed: Jun. 20, 2024

Related U.S. Application Data
(63) Continuation of application No. PCT/CN2024/071489, filed on Jan. 10, 2024.

(30) Foreign Application Priority Data
Aug. 29, 2023 (CN) 202311093260.3

(51) Int. Cl. C12M 1/00; C12M 1/34; C12M 3/00

(52) U.S. Cl. CPC C12M 29/04; C12M 23/34; C12M 23/38; C12M 23/48; C12M 41/32

(57) ABSTRACT

A highly-breathable closed cell culture device includes: a base; a reactor housing, located above the base and connected to the base to form a closed chamber; a gas exchange assembly, located above the base and communicated with the closed chamber; a first liquid exchange assembly, located above the reactor housing and connected to the reactor housing; and a second liquid exchange assembly, located on a side wall of the reactor housing and communicated with the closed chamber. A filter is provided in the first liquid exchange assembly, and a filtration membrane is built in the filter in a folded or stacked manner. A gas permeation rate of the filter is 5 L/min-30 L/min, and an area of the filtration membrane is greater than 2,700 mm². The culture device solves the problems that the bottom breathable silicone membrane bulges due to negative pressure when pumping the culture medium.

### CROSS REFERENCE TO THE RELATED APPLICATIONS

This application is the continuation application of International Application No. PCT/CN2024/071489, filed on Jan. 10, 2024, which is based upon and claims priority to Chinese Patent Application No. 202311093260.3, filed on Aug. 29, 2023, the entire contents of which are incorporated herein by reference.

### TECHNICAL FIELD

The present disclosure belongs to the technical field of cell culture, and in particular to a highly-breathable closed cell culture device and method.

### BACKGROUND

Cell culture refers to the technology of cell growth and proliferation in vitro through necessary nutrients and gases. With the development of pharmaceutical technology, cell culture technology has increasingly become an essential technical means in pharmaceutical research and drug production. The current cell culture technology has gradually been applied in many technical fields such as drug screening research, monoclonal antibody production, vaccine production, and cell therapy.

In the prior art, cells used for T-cell therapy, adoptive immunotherapy, and adoptive cell therapy are often prepared and stored using conventional methods. However, due to the limited space and nutrients for cell growth, passaging is required when the number of cultured cells reaches a certain value. Usually, when the number of cells increases significantly, it is necessary to add the culture medium frequently or separate cells from the culture medium, during which a large amount of cell consumables are used to increase the cell number. However, on one hand, this operating method requires repeated opening of the lid, which greatly increases the risk of cell contamination during the cell preparation process and prolongs the cell harvest time. Once cells are contaminated by bacteria, they will stop proliferation and die in large numbers, causing huge losses. On the other hand, cell growth requires carbon dioxide and oxygen. Existing culture devices have low gas permeation efficiency and do not allow for the addition of a large amount of culture medium at once, making it hard to achieve long-term cell culture. This ultimately leads to low cell culture efficiency and a significant increase in cell culture costs.

Cell and gene therapy (CGT) refers to the process of transferring an identified genetic material to specific target cells of a patient and modifying individual gene expression or repairing abnormal genes through gene addition, gene modification, gene silencing, etc., in order to cure a disease. According to the American Society of Cell and Gene Therapy (ASCGT), over 3,600 clinical trials are currently underway in the online pipeline of CGT worldwide. So far, 23 gene and cell therapy products have been approved by the Food and Drug Administration (FDA), including disruptive therapies for diseases such as neuromuscular disorders, hereditary blindness, and certain blood cancers. In China, 726 trials have been conducted and 5 drugs have been launched. CGT has high targeting ability and can kill cancer cells without damaging normal cells. In addition, since the cells originate from the patient's body, CGT prevents immunological rejection and has basically no side effects or adverse reactions, so patients will not feel pain during the treatment process. CGT requires extracting defective cells from the patient's body for genetic modification, conducting cell culture and proliferation, and returning cultured cells to the patient's body. The conventional cell culture process uses a conventional cell culture flask. Due to the limited number of cells growing in a single consumable culture, it is necessary to regularly open the lid for cell passage or detection, which can easily cause cell contamination. Therefore, this culture method is inevitably not suitable for CGT cell culture. CGT cell culture and proliferation should be carried out as much as possible in a completely closed system to reduce unforeseeable contamination caused by human intervention.

To address the above-mentioned issues, there is an urgent need for a highly-breathable fully-closed cell culture system. The entire process from cell culture to collection is carried out in the fully-closed system, and cells and culture medium are transported through pipelines, which greatly reduces the risk of contamination.

Chinese patent application CN113462570A provides a large-capacity cell suspension culture flask. Specifically, the large-capacity cell suspension culture flask is a fully closed operating device that uses an integrated culture flask to reduce the risk of cross contamination during the cell culture process. The culture flask exchanges culture medium through pumping. The upper breathable membrane body filters the gas through a conventional single-layer membrane filter, which features a small number of pores per unit area and low gas permeation rate, suitable for gas exchange during cell culture. However, it cannot maintain real-time and instantaneous pressure balance inside and outside the flask during medium pumping. Pumping the culture medium may cause negative pressure inside the flask, causing the bottom breathable silicone membrane to bulge, thereby affecting the cells being cultured. As a result, the cells close to the lower connecting pipe will be disturbed and pumped out together with the waste liquid, resulting in a decrease in cell culture efficiency and yield. In addition, due to the low gas permeation rate of the upper breathable membrane body, the supplementation of the culture medium is slow, affecting cell growth.

In order to avoid negative pressure inside the flask caused by pumping of the culture medium, Chinese patent application CN105392876A provides a breathable closed cell culture system device and method. The closed system device utilizes pressure to push the gas to the cell culture and recovery device, thereby discharging the culture medium and cultured cells. Specifically, an air pump is connected to introduce sterile air into the flask so as to create positive pressure for discharging the liquid. However, the pressurization timing is difficult to control, requiring gas flow control and monitoring to determine whether the liquid is fully discharged, which increases the complexity of the system.

In order to solve the above-mentioned technical problems, it is necessary to provide a highly-breathable closed cell culture device and method.

### SUMMARY

### Technical Problems

The present disclosure aims to solve the problems in prior art, that is, the bottom breathable silicone membrane bulges due to negative pressure when pumping the culture medium, and the positive pressure method for discharging the culture medium makes the equipment complex.

### Technical Solutions

In order to solve the above technical problems, the present disclosure provides the following technical solutions.

A first aspect of the present disclosure provides a highly-breathable closed cell culture device, including: a base; a reactor housing, located above the base and connected to the base to form a closed chamber; a gas exchange assembly, located above the base and communicated with the closed chamber; a first liquid exchange assembly, located above the reactor housing and connected to the reactor housing; and a second liquid exchange assembly, located on a side wall of the reactor housing and communicated with the closed chamber; where a filter is provided in the first liquid exchange assembly; a filtration membrane is provided in the filter and is built in the filter in a folded or stacked manner; and a gas permeation rate of the filter is 5 L/min-30 L/min, and an area of the filtration membrane is greater than 2,700 mm².

Further, the first liquid exchange assembly includes: a communicating member, located at a top of the reactor housing and connected to the reactor housing; an end cap, located above the communicating member and connected to the communicating member, thereby limiting, through the communicating member, the closed chamber from being communicated with an outside world; a liquid communicating pipe, inserted inside the end cap and connected to the end cap; and a gas communicating pipe, inserted inside the end cap and connected to the end cap, where the filter is provided in the gas communicating pipe.

Further, a pore size of the filtration membrane is not greater than 0.22 μm, and the filtration membrane is made of any one of polypropylene (PP), polyether sulfone (PES), polytetrafluoroethylene (PTFE), and a nanofiltration (NF) material.

Further, the gas exchange assembly includes: a support net, located above the base and connected to the base; a sealing ring, located above the support net and connected to the support net; and a second breathable membrane, located between the support net and the sealing ring and connected to the support net and the sealing ring.

Further, the second breathable membrane is a hydrophobic breathable membrane, allowing the passage of a gas required for cell growth and blocking the flow of a culture medium; and the second breathable membrane is made of silicone.

Further, the second liquid exchange assembly includes: a discharge outlet, located on the side wall of the reactor housing and communicated with the closed chamber; and a discharge pipe, located at the discharge outlet and connected to the discharge outlet.

Optionally, the culture device further includes a first breathable membrane, and the first breathable membrane is located above the reactor housing and communicated with the closed chamber.

Optionally, the culture device further includes a partition assembly, located inside the reactor housing and connected to an inner side wall of the reactor housing to divide the closed chamber into a first chamber and a second chamber. The first chamber is located above the second chamber; and a volume ratio between the first chamber and the second chamber is 9:1.

Further, the partition assembly includes: a clamping slot, located on a circumference of an inner wall of the reactor housing and connected to the inner wall of the reactor housing; a clamping plate, where an edge of the clamping plate is located inside the clamping slot and connected to the clamping slot to divide the chamber inside the reactor housing into the first chamber and the second chamber; an exchange port, passing through the clamping plate, where the first chamber and the second chamber are communicated with each other through the exchange port; and a semi-permeable membrane, located on the clamping plate and connected to the clamping plate, where the semi-permeable membrane allows the passage of a substance with a molecular weight less than 10 kDa.

The present disclosure further provides a cell culture method, implemented by the highly-breathable closed cell culture device, and specifically including the following steps:

### S1: Culture Medium Addition:

adding a culture medium to the closed chamber of the reactor housing through the liquid communicating pipe;

### S2: Cell Inoculation:

adding a target cell to the closed chamber of the reactor housing through the liquid communicating pipe;

### S3: Cell Culture:

keeping the gas exchange assembly unobstructed during an entire process of cell culture to ensure the supply of a gas required for cell growth, and carrying out the cell culture in an incubator; and taking, during the culture process, a sample through the liquid communicating pipe to monitor a change in a nutrient in the culture medium, and adding a missing nutrient;

### S4: Culture Medium Discharge:

communicating the liquid communicating pipe with a waste liquid flask, and discharging a waste culture medium from the closed chamber through a peristaltic pump; and allowing, during a discharge process, external air to enter the closed chamber after the external air passes through the filter to filter out a contaminant, thereby ensuring pressure balance inside the device; and

### S5: Cell Collection:

collecting cells through the second liquid exchange assembly.

The present disclosure further provides another cell culture method, implemented by the highly-breathable closed cell culture device, and specifically including the following steps:

### S1: Cell Inoculation:

diluting required cells with a culture medium that accounts for 10% of a volume of the closed chamber, and adding the cells to the second chamber of the reactor housing through the second liquid exchange assembly;

### S2: Culture Medium Addition:

adding the culture medium to the first chamber through the liquid communicating pipe, where the added culture medium accounts for 90% of the volume of the closed chamber;

### S3: Cell Culture:

keeping the first breathable membrane and the gas exchange assembly unobstructed during an entire process of cell culture to ensure the supply of a gas required for cell growth, and carrying out the cell culture in an incubator;

### S4: Culture Medium Replacement:

pumping out, in case of a need to replace the culture medium, a waste culture medium through the liquid communicating pipe; allowing, during a pumping process, external air to enter the closed chamber after the external air passes through the filter to filter out a contaminant, so as to ensure pressure balance inside the device; and feeding, after the pumping is completed, a fresh culture medium through the liquid communicating pipe for cell growth; and

### S5: Concentrated Cell Collection:

repeating the step S4 for multiple times to obtain multiple batches of mixed cultured cells, cell secretion product and culture medium in the second chamber, to obtain concentrated cells; communicating the first liquid exchange assembly with a waste liquid flask, and discharging the waste culture medium from the first chamber; mixing the cultured cells, the cell secretion product and the culture medium in the second chamber, and allowing the cultured cells and the cell secretion product to suspend in the culture medium; and opening the second liquid exchange assembly, and transferring and collecting the cultured cells, the cell secretion product and the culture medium through the second liquid exchange assembly.

### Beneficial Effects

The present disclosure proposes a highly-breathable closed cell culture device and method. The present disclosure aims to solve the problems in prior art, that is, the bottom breathable silicone membrane bulges due to negative pressure when pumping the culture medium, and the positive pressure method for discharging the culture medium makes the equipment complex. Therefore, the present disclosure has practical value.

### BRIEF DESCRIPTION OF THE DRAWINGS

FIG. 1 is an exploded view of a device according to the present disclosure;

FIG. 2 is an assembled view of the device according to the present disclosure;

FIG. 3 is an internal view of the device according to the present disclosure; and

FIG. 4 is another internal view of the device according to the present disclosure.

### REFERENCE NUMERALS

1. first breathable membrane; 2. gas exchange assembly; 2-1. sealing ring; 2-2. second breathable membrane; 2-3. support net; 3. first liquid exchange assembly; 3-1. end cap; 3-2. communicating member; 3-3. liquid communicating pipe; 3-4. gas communicating pipe; 3-5. filter; 4. second liquid exchange assembly; 4-1. discharge outlet; 4-2. discharge pipe; 5. base; 6. partition assembly; 6-1. clamping slot; 6-2. clamping plate; 6-3. exchange port; 6-4. semi-permeable membrane; 7. reactor housing; 7-1. first chamber; and 7-2. second chamber.

### DETAILED DESCRIPTION OF THE EMBODIMENTS

Exemplary implementations of the present disclosure are described below in further detail with reference to the drawings. Although the drawings show exemplary implementations of the present disclosure, it should be understood that the present disclosure can be implemented in various forms and should not be limited to the implementations set forth herein. On the contrary, these implementations are provided, such that the present disclosure is fully understandable and the scope of the present disclosure is fully conveyed to those skilled in the art. Unless otherwise specified, the technical means used in the embodiments are conventional means well known to those skilled in the art.

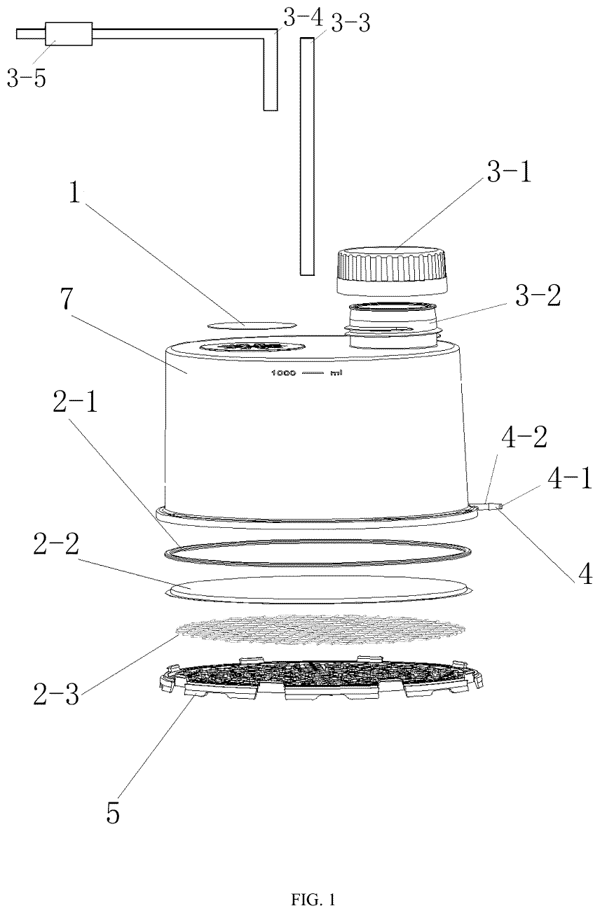

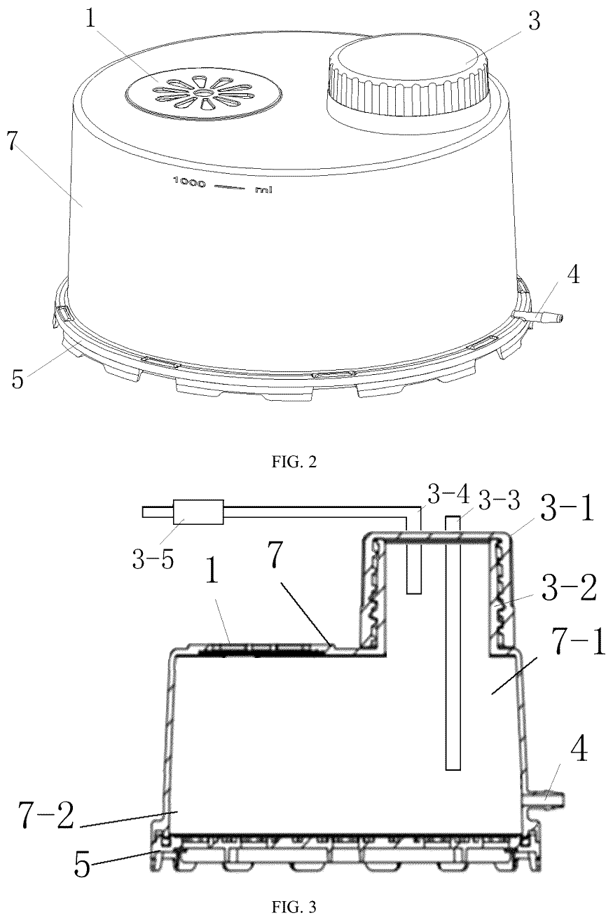

As shown in FIGS. 1 to 3, an embodiment provides a highly-breathable closed cell culture device. The device includes: base 5, reactor housing 7, gas exchange assembly 2, first liquid exchange assembly 3, and second liquid exchange assembly 4.

The reactor housing 7 is located above the base 5 and connected to the base 5 to form a closed space, thereby isolating external air.

As shown in FIG. 1, the gas exchange assembly 2 is located above the base 5 and communicated with the enclosed space or second chamber 7-2. Specifically, the gas exchange assembly 2 includes: support net 2-3 located above the base 5 and connected to the base 5; sealing ring 2-1 located above the support net 2-3 and connected to the support net 2-3; and second breathable membrane 2-2 located between the support net 2-3 and the sealing ring 2-1 and connected to the support net 2-3 and the sealing ring 2-1. The second breathable membrane 2-2 is a hydrophobic breathable membrane. Cells can directly obtain the gas required for cell growth from the second breathable membrane 2-2 located at the bottom, improving the gas transfer rate. The second breathable membrane 2-2 can block the flow of culture medium, and the second breathable membrane 2-2 is made of silicone. Cells will naturally settle on the second breathable membrane 2-2 during the static suspension culture process.

As shown in FIGS. 1 to 3, the first liquid exchange assembly 3 is located above the reactor housing 7, adjacent to first breathable membrane 1, and connected to the reactor housing 7 for feeding and/or pumping the culture medium.

Components of the first liquid exchange assembly 3 are described below.

Communicating member 3-2 is located at a top of the reactor housing 7 and connected to the reactor housing 7. The communicating member 3-2 is provided with a top open end, and an outer circumference of the open end is provided with a thread. The communicating member is threaded to end cap 3-1.

The end cap 3-1 is located above the communicating member 3-2 and threaded to the communicating member 3-2, thereby limiting a closed chamber from being communicated with an outside world through the communicating member 3-2. A rubber pad is provided between the end cap 3-1 and the communicating member 3-2 to ensure the sealing effect.

Liquid communicating pipe 3-3 is inserted inside the end cap 3-1 and connected to the end cap 3-1, such that the closed chamber is communicated with the outside through the communicating pipe 3-3.

Gas communicating pipe 3-4 is inserted inside the end cap 3-1, adjacent to the liquid communicating pipe 3-3, and connected to the end cap 3-1.

The liquid communicating pipe 3-3 is inserted inside the communicating member 3-2 and connected to the communicating member 3-2, such that first chamber 7-1 is communicated with an external waste culture medium recovery device or fresh culture holding device through the liquid communicating pipe 3-3. A peristaltic pump is provided on the communicating pipe to provide pumping pressure. Compared to the air pump described in Chinese patent application CN105392876A, the pressure generated by the peristaltic pump is much smaller and will not have a destructive effect on the interior of the reactor. The liquid communicating pipe 3-3 is sealed and closed when not in use. There may be one, two, or more liquid communicating pipes 3-3 provided. When there is only one liquid communicating pipe 3-3, the liquid communicating pipe realizes the feeding and pumping of the culture medium. When there are two liquid communicating pipes 3-3, one liquid communicating pipe 3-3 is configured to feed the culture medium, and the other liquid communicating pipe is configured to pump the culture medium. The liquid communicating pipe 3-3 includes one end threaded through the end cap 3-1 to facilitate connection with the pipeline of the peristaltic pump and the other end located at a height of 10% volume of the reactor housing 7.

The gas communicating pipe 3-4 is inserted inside the end cap 3-1, adjacent to the liquid communicating pipe 3-3, and connected to the end cap 3-1. Filter 3-5 is provided outside the reactor housing 7 and connected to gas communicating pipes 3-4.

The filter 3-5 is located outside the reactor housing 7 and connected to the gas communicating pipe 3-4. A filtration membrane is provided in the filter 3-5. The filtration membrane is placed inside the filter 3-5 in a folded or stacked manner. A gas permeation rate of the filter 3-5 is 5 L/min-30 L/min, an area of the filtration membrane is greater than 2,700 mm², and a pore size of 0.22 μm. The filtration membrane is made of any one of polypropylene (PP), polyether sulfone (PES), polytetrafluoroethylene (PTFE), and a nanofiltration (NF) material. When the culture medium or cells are discharged by pumping, external air is passed through the filter 3-5 to provide sterile air to the interior of the closed chamber, ensuring pressure balance inside the closed chamber.

Conventional syringe filters belong to the membrane type, with a small filter area and low gas permeation rate, which can easily form negative pressure inside the cell culture flask, making it difficult to transfer liquid and causing the bottom breathable membrane to bulge. In the present disclosure, the device is externally connected to the high-breathability filter 3-5. The filter 3-5 has antibacterial and breathable functions, and a large area of folded or stacked multi-layer filtration membrane structure is provided in the filter 3-5. The folded or stacked multi-layer filtration membrane has a large area and a large porosity per unit area. The increase in the gas permeation rate involves an increase in the membrane area and an exponential increase in the number of pores per unit area. Therefore, compared to conventional membrane filters, the device has significant advantages in gas permeation rate. In this device, the gas permeation rate of the filter 3-5 can reach 5 L/min-30 L/min, while the maximum gas permeation rate of conventional membrane filters is only 0.05 L/min. In terms of area, the area of a 25 mm membrane filter is 490 mm², and the area of the filter 3-5 with the same diameter can reach 2,732 mm² to 16,200 mm². During the cell collection process, this device greatly improves the instantaneous gas permeation rate due to the filter 3-5. When the culture medium or cells are discharged by pumping, the filter 3-5 can quickly provide sterile air, reducing the negative pressure inside the flask caused by pumping of the culture medium, thereby maintaining pressure balance inside the reactor. When pumping the culture medium, the second breathable membrane 2-2 (breathable silicone membrane) located at the bottom will not bulge due to negative pressure, avoiding cell loss during pumping and preventing the rupture of the breathable membrane, thereby preventing culture medium leakage and contamination. Meanwhile, this device does not need to be connected to an air pump and directly uses the peristaltic pump to pump out the liquid. Due to the increase in the breathable area of the filter 3-5, the liquid discharge speed increases and the operation time is shortened. Air enters the culture flask directly through the filter 3-5, achieving easy and convenient use.

As shown in FIG. 1, the second liquid exchange assembly 4 is located on a side wall of the reactor housing 7 and communicated with the closed chamber to achieve cell feeding and/or discharging.

Specifically, the second liquid exchange assembly 4 includes: discharge outlet 4-1 located on the side wall of the reactor housing 7 and communicated with the closed chamber of the reactor housing 7; and discharge pipe 4-2 located at the discharge outlet 4-1 and connected to the discharge outlet 4-1. A collection flask can be connected through a pipeline, namely a cell collection pipeline. A distance from the discharge outlet 4-1 to a bottom defines a height that is one tenth of an effective volume of a flask body. The discharge pipe 4-2 is sealed and closed when not in use.

The first breathable membrane 1 is made of PTFE and located above the reactor housing 7 to increase the gas exchange efficiency during the culture process.

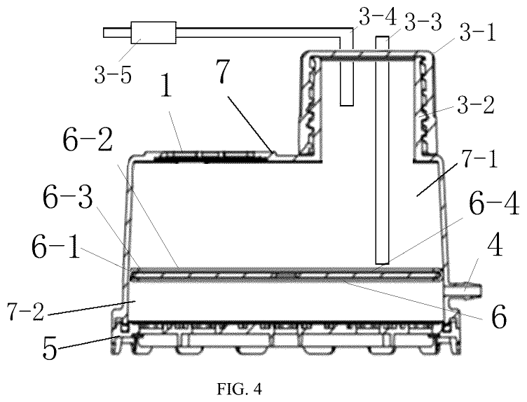

Preferably, as shown in FIG. 4, this device may further include partition assembly 6. The partition assembly 6 is provided inside the reactor housing 7 and connected to an inner side wall of the reactor housing 7 to divide the interior of the reactor housing 7 into the first chamber 7-1 and the second chamber 7-2. The first chamber 7-1 is located above the second chamber 7-2. In this embodiment, the partition assembly 6 avoids cell disturbance during the discharge of waste culture medium, thereby avoiding cell loss caused by the discharge of waste culture medium. In this way, the device achieves effective separation of culture medium and cultured cells. The device can discharge waste culture medium and add fresh culture medium in the closed space without affecting cell growth. The device can add fresh culture medium under sterile conditions to ensure long-term uninterrupted cell culture.

Further, the partition assembly 6 includes: clamping slot 6-1, clamping plate 6-2, and exchange port 6-3. The clamping slot is located on a circumference of an inner wall of the reactor housing 7 and connected to the inner wall of the reactor housing 7. An edge of the clamping plate 6-2 is located inside the clamping slot 6-1 and connected to the clamping slot 6-1 to divide the chamber inside the reactor housing 7 into the first chamber 7-1 and the second chamber 7-2. The clamping plate 6-2 can include one or more layers. The clamping plate 6-2 has a circular or net structure. The exchange port passes through the clamping plate 6-2. The first chamber 7-1 and the second chamber 7-2 are communicated with each other through the exchange port 6-3. When the clamping plate 6-2 is in a circular structure, a middle region of the circular structure forms the exchange port 6-3. When the clamping plate 6-2 is in a net structure, a net hole forms the exchange port 6-3. The area of the exchange port 6-3 should be as large as possible to meet the exchange efficiency of the culture medium.

The partition assembly further includes semi-permeable membrane 6-4. When the clamping plate 6-2 includes one layer, the semi-permeable membrane 6-4 is fixed to the clamping plate 6-2 and connected to the clamping plate 6-2. When the clamping plate 6-2 includes two or more layers, the semi-permeable membrane 6-4 is clamped and fixed between the two or more layers of the clamping plate 6-2. Nutrients with a molecular weight less than 10 kDa can penetrate the semi-permeable membrane 6-4 of the partition assembly 6 and penetrate from the first chamber 7-1 to the second chamber 7-2. Metabolic inhibitors penetrate from the second chamber 7-2 to the first chamber 7-1, and all target proteins and cells remain in the second chamber 7-2. The semi-permeable membrane 6-4 can block cells, as well as antibodies and proteins with high molecular weight, and allow free exchange of the culture medium. Preferably, a volume ratio between the first chamber 7-1 and the second chamber 7-2 is 9:1. That is, the partition assembly 6 is located at a height that is 10% of the volume above the bottom of the culture flask. Since cells will naturally settle at the bottom of the device during static suspension culture, the first chamber 7-1 is configured to hold only the culture medium, thereby achieving rich culture medium and nutrient supply. The second chamber 7-2 is configured to hold 10% volume of culture medium and cultured cells, forming a high-density cell culture region and concentrating the produced antibodies and proteins.

Another aspect of the present disclosure proposes a method for the highly-breathable closed cell culture device. When the partition assembly 6 is not provided, the method is suitable for operations in CGT, and the method specifically includes the following steps.

### S1: Culture Medium Addition.

The culture medium is added to the closed chamber of the reactor housing 7 through the liquid communicating pipe 3-3, where the added culture medium accounts for 90% of the volume of the closed chamber.

### S2: Cell Inoculation.

Required cells are diluted with culture medium that accounts for 10% of the volume of the closed chamber, and are added to the closed chamber of the reactor housing 7 through the liquid communicating pipe 3-3.

### S3: Cell Culture.

During the entire process of cell culture, the gas exchange assembly 2 is unobstructed to ensure the supply of the gas required for cell growth, and the cell culture is carried out in an incubator. During the culture process, samples are taken through the liquid communicating pipe 3-3 to monitor changes in nutrients in the culture medium and add missing nutrients.

### S4: Culture Medium Discharge.

The liquid communicating pipe 3-3 is communicated with a waste liquid flask, and waste culture medium is discharged from the closed chamber through the peristaltic pump. During the discharge process, external air is passed through the filter 3-5 (to filter out contaminants) to enter the closed chamber, so as to quickly ensure pressure balance inside the device.

### S5: Cell Collection.

Cells are collected through the second liquid exchange assembly 4.

When the partition assembly 6 is provided, this device is suitable for operations related to CGT, in particular for concentration and collection of antibodies and proteins. In this device, the second breathable membrane 2-2 and the middle semi-permeable membrane 6-4 break through the limitations of conventional containers such as culture plates, square flasks, and culture bags in terms of cultivation height. The static cultivation height of conventional culture plates and square flasks is about 0.3 cm, and the height of culture bags is about 1.0 cm. If the cultivation height exceeds the limit height, the oxygen and nutrient supply inside the culture device will be greatly limited, making it impossible to ensure sufficient oxygen and nutrients required for cell culture. In addition, the waste gas generated by cell growth cannot be discharged, resulting in a sharp decrease in cell growth viability and yield. The cultivation height of this device is approximately 10 cm.

For conventional culture plates and square flasks, when the cultured cells produce proteins or antibodies, due to the volume and nutrient supply limitations of the culture medium, static culture can generally only last for about 3-5 days. This device breaks through the limitation of conventional cultivation containers in terms of cultivation height. The second breathable membrane 2-2 at the bottom can balance oxygen and carbon dioxide. The semi-permeable membrane 6-4 allows the passage of substances with a molecular weight less than 10 kDa to exchange fresh culture medium and metabolites with the second chamber 7-2 for cell culture, while retaining all proteins in the second chamber 7-2. According to the user's experimental design, cell culture can last for about 10-14 days, greatly extending the cell culture time and producing enough proteins and antibodies. For conventional culture containers, when collecting cells and antibodies or proteins, the entire culture medium, cells, proteins, and antibodies are collected together. In this device, the semi-permeable membrane 6-4 suitable for a molecular weight of about 10 kDa concentrates the generated antibodies and proteins in the second chamber 7-2 of the membrane, and collects those that account for about 1/10 of the volume in conventional cell culture methods, greatly reducing downstream workload.

The specific steps of this method are as follows.

### S1: Cell Inoculation.

Required cells are diluted with culture medium that accounts for 10% of the volume of the closed chamber, and are added to the second chamber 7-2 of the reactor housing 7 through the second liquid exchange assembly 4.

### S2: Culture Medium Addition.

The culture medium is added to the first chamber 7-1 through the liquid communicating pipe 3-3, where the added culture medium accounts for 90% of the volume of the closed chamber.

### S3: Cell Culture.

During the entire process of cell culture, the first breathable membrane and the gas exchange assembly 2 are unobstructed to ensure the supply of the gas required for cell growth, and the cell culture is carried out in an incubator.

### S4: Culture Medium Replacement.

When it is necessary to replace the culture medium, waste culture medium is pumped out through the liquid communicating pipe 3-3. During the pumping process, external air is passed through the filter 3-5 (to filter out contaminants) to enter the closed chamber, so as to quickly ensure the pressure balance inside the device. After the pumping is completed, a fresh culture medium is fed through the liquid communicating pipe 3-3 for cell growth.

### S5: Concentrated Cell Collection.

The step S4 is repeated for multiple times to obtain multiple batches of mixed cultured cells, cell secretion products, and culture medium in the second chamber 7-2, to obtain concentrated cells. The first liquid exchange assembly 3 is communicated with the waste liquid flask, and the waste culture medium is discharged from the first chamber 7-1. The cultured cells, the cell secretion products and the culture medium are mixed in the second chamber 7-2, and the cultured cells and the cell secretion products are suspended in the culture medium. The second liquid exchange assembly is opened, and the cultured cells, cell secretion products, and culture medium are transferred and collected through the second liquid exchange assembly 4.

This device can cultivate cells with high density, while providing sufficient nutrition and a long cultivation time. In this device, the antibodies and proteins produced by cell culture are greatly more than those produced using conventional culture methods. For example, for hybridoma, the antibodies produced have a concentration around 1-5 mg/mL, which is 50-100 times more than those produced using conventional culture methods.

### Implementations of the Present Disclosure

When the partition assembly 6 is not provided, the method is suitable for operations in CGT, and the method specifically includes the following steps.

### S1: Culture Medium Addition.

The culture medium is added to the closed chamber of the reactor housing 7 through the liquid communicating pipe 3-3, where the added culture medium accounts for 90% of the volume of the closed chamber.

### S2: Cell Inoculation.

Required cells are diluted with culture medium that accounts for 10% of the volume of the closed chamber, and are added to the closed chamber of the reactor housing 7 through the liquid communicating pipe 3-3.

### S3: Cell Culture.

During the entire process of cell culture, the gas exchange assembly 2 is unobstructed to ensure the supply of the gas required for cell growth, and the cell culture is carried out in an incubator. During the culture process, samples are taken through the liquid communicating pipe 3-3 to monitor changes in nutrients in the culture medium and add missing nutrients.

### S4: Culture Medium Discharge.

The liquid communicating pipe 3-3 is communicated with a waste liquid flask, and waste culture medium is discharged from the closed chamber through the peristaltic pump. During the discharge process, external air is passed through the filter 3-5 (to filter out contaminants) to enter the closed chamber, so as to quickly ensure pressure balance inside the device.

### S5: Cell Collection.

Cells are collected through the second liquid exchange assembly 4.

When the partition assembly 6 is provided, this device is suitable for operations related to CGT, in particular for concentration and collection of antibodies and proteins. In this device, the second breathable membrane 2-2 and the middle semi-permeable membrane 6-4 break through the limitations of conventional containers such as culture plates, square flasks, and culture bags in terms of cultivation height. The static cultivation height of conventional culture plates and square flasks is about 0.3 cm, and the height of culture bags is about 1.0 cm. If the cultivation height exceeds the limit height, the oxygen and nutrient supply inside the culture device will be greatly limited, making it impossible to ensure sufficient oxygen and nutrients required for cell culture. In addition, the waste gas generated by cell growth cannot be discharged, resulting in a sharp decrease in cell growth viability and yield. The cultivation height of this device is approximately 10 cm.

For conventional culture plates and square flasks, when the cultured cells produce proteins or antibodies, due to the volume and nutrient supply limitations of the culture medium, static culture can generally only last for about 3-5 days. This device breaks through the limitation of conventional cultivation containers in terms of cultivation height. The second breathable membrane 2-2 at the bottom can balance oxygen and carbon dioxide. The semi-permeable membrane 6-4 allows the passage of substances with a molecular weight less than 10 kDa to exchange fresh culture medium and metabolites with the second chamber 7-2 for cell culture, while retaining all proteins in the second chamber 7-2. According to the user's experimental design, cell culture can last for about 10-14 days, greatly extending the cell culture time and producing enough proteins and antibodies. For conventional culture containers, when collecting cells and antibodies or proteins, the entire culture medium, cells, proteins, and antibodies are collected together. In this device, the semi-permeable membrane 6-4 suitable for a molecular weight of about 10 kDa concentrates the generated antibodies and proteins in the second chamber 7-2 of the membrane, and collects those that account for about 1/10 of the volume in conventional cell culture methods, greatly reducing downstream workload.

The specific steps of this method are as follows.

### S1: Cell Inoculation.

Required cells are diluted with culture medium that accounts for 10% of the volume of the closed chamber, and are added to the second chamber 7-2 of the reactor housing 7 through the second liquid exchange assembly 4.

### S2: Culture Medium Addition.

The culture medium is added to the first chamber 7-1 through the liquid communicating pipe 3-3, where the added culture medium accounts for 90% of the volume of the closed chamber.

### S3: Cell Culture.

During the entire process of cell culture, the first breathable membrane and the gas exchange assembly 2 are unobstructed to ensure the supply of the gas required for cell growth, and the cell culture is carried out in an incubator.

### S4: Culture Medium Replacement.

When it is necessary to replace the culture medium, waste culture medium is pumped out through the liquid communicating pipe 3-3. During the pumping process, external air is passed through the filter 3-5 (to filter out contaminants) to enter the closed chamber, so as to quickly ensure the pressure balance inside the device. After the pumping is completed, a fresh culture medium is fed through the liquid communicating pipe 3-3 for cell growth.

### S5: Concentrated Cell Collection.

The step S4 is repeated for multiple times to obtain multiple batches of mixed cultured cells, cell secretion products, and culture medium in the second chamber 7-2, to obtain concentrated cells. The first liquid exchange assembly 3 is communicated with the waste liquid flask, and the waste culture medium is discharged from the first chamber 7-1. The cultured cells, the cell secretion products and the culture medium are mixed in the second chamber 7-2, and the cultured cells and the cell secretion products are suspended in the culture medium. The second liquid exchange assembly is opened, and the cultured cells, cell secretion products, and culture medium are transferred and collected through the second liquid exchange assembly 4.

### INDUSTRIAL APPLICABILITY

The present disclosure proposes a highly-breathable closed cell culture device and method. In the present disclosure, the device is externally connected to a high-breathability filter. A large area of folded or stacked multi-layer filtration membrane structure is provided in the filter. The folded or stacked multi-layer filtration membrane not only increases the membrane area, but also increases the number of pores per unit area exponentially. Therefore, compared to conventional membrane filters, the device has significant advantages in gas permeation rate. This device greatly improves the instantaneous gas permeation rate, and reduces the negative pressure inside the flask caused by pumping of the culture medium, preventing the breathable silicone membrane at the bottom from bulging due to negative pressure when pumping the culture medium. Meanwhile, compared to a device that uses positive pressure to discharge culture medium, the device in the present disclosure does not need to be connected to an air pump and directly uses the peristaltic pump to pump out the liquid. Since the filter balances pressure in the closed space, the liquid discharge speed increases and the operation time is shortened. Air enters the closed culture space directly through the filter, achieving easy and convenient use.

In the present disclosure, the device further includes a partition assembly. The partition assembly avoids cell disturbance during the discharge of waste culture medium, thereby avoiding cell loss caused by the discharge of waste culture medium. In this way, the device achieves effective separation of culture medium and cultured cells. The device can discharge waste culture medium and add fresh culture medium in the closed space without affecting cell growth, thereby ensuring long-term uninterrupted cell culture. The device forms a high-density cell culture region, and concentrates the produced antibodies and proteins.

This device can cultivate cells with high density, while providing sufficient nutrition and a long cultivation time. In this device, the antibodies and proteins produced by cell culture are greatly more than those produced using conventional culture methods. For example, for hybridoma, the antibodies produced have a concentration around 1-5 mg/mL, which is 50-100 times more than those produced using conventional culture methods.

What is claimed is:

1. A highly-breathable closed cell culture device, comprising:
a base;
a reactor housing, located above the base and connected to the base to form a closed chamber;
a gas exchange assembly, located above the base and communicated with the closed chamber;
a first liquid exchange assembly, located above the reactor housing and connected to the reactor housing; and
a second liquid exchange assembly, located on a side wall of the reactor housing and communicated with the closed chamber;
wherein, a filter is provided in the first liquid exchange assembly; a filtration membrane is built in the filter in a folded or stacked manner; and a gas permeation rate of the filter is 5 L/min-30 L/min, and an area of the filtration membrane is greater than 2,700 mm².

2. The highly-breathable closed cell culture device according to claim 1, wherein the first liquid exchange assembly comprises:
a communicating member, located at a top of the reactor housing and connected to the reactor housing;
an end cap, located above the communicating member and connected to the communicating member, wherein the end cap is configured to limit, through the communicating member, the closed chamber from being communicated with an outside world;
a liquid communicating pipe, inserted inside the end cap and connected to the end cap; and
a gas communicating pipe, inserted inside the end cap and connected to the end cap, wherein the filter is provided in the gas communicating pipe.

3. The highly-breathable closed cell culture device according to claim 1, wherein a pore size of the filtration membrane is less than or equal to 0.22 μm, and the filtration membrane is made of any one selected from the group consisting of polypropylene (PP), polyether sulfone (PES), polytetrafluoroethylene (PTFE), and a nanofiltration (NF) material.

4. The highly-breathable closed cell culture device according to claim 1, wherein the gas exchange assembly comprises:
a support net, located above the base and connected to the base;
a sealing ring, located above the support net and connected to the support net; and
a second breathable membrane, located between the support net and the sealing ring and connected to the support net and the sealing ring.

5. The highly-breathable closed cell culture device according to claim 4, wherein the second breathable membrane is a hydrophobic breathable membrane, and the second breathable membrane is configured to allow a passage of a gas required for cell growth and block a flow of a culture medium; and the second breathable membrane is made of silicone.

6. The highly-breathable closed cell culture device according to claim 1, wherein the second liquid exchange assembly comprises:
a discharge outlet, located on the side wall of the reactor housing and communicated with the closed chamber; and
a discharge pipe, located at the discharge outlet and connected to the discharge outlet.

7. The highly-breathable closed cell culture device according to claim 1, further comprising a first breathable membrane, wherein the first breathable membrane is located above the reactor housing and communicated with the closed chamber.

8. The highly-breathable closed cell culture device according to claim 1, further comprising:
a partition assembly, located inside the reactor housing and connected to an inner side wall of the reactor housing to divide the closed chamber into a first chamber and a second chamber, wherein the first chamber is located above the second chamber; and a volume ratio between the first chamber and the second chamber is 9:1.

9. The highly-breathable closed cell culture device according to claim 8, wherein the partition assembly comprises:
a clamping slot, located on a circumference of an inner wall of the reactor housing and connected to the inner wall of the reactor housing;
a clamping plate, wherein an edge of the clamping plate is located inside the clamping slot and connected to the clamping slot to divide the closed chamber inside the reactor housing into the first chamber and the second chamber;
an exchange port, passing through the clamping plate, wherein the first chamber and the second chamber are communicated with each other through the exchange port; and
a semi-permeable membrane, located on the clamping plate and connected to the clamping plate, wherein the semi-permeable membrane allows a passage of a substance with a molecular weight less than 10 kDa.

10. A cell culture method, implemented by a highly-breathable closed cell culture device comprising:
a base;
a reactor housing, located above the base and connected to the base to form a closed chamber:
a gas exchange assembly, located above the base and communicated with the closed chamber;
a first liquid exchange assembly, located above the reactor housing and connected to the reactor housing; and
a second liquid exchange assembly, located on a side wall of the reactor housing and communicated with the closed chamber;
wherein, a filter is provided in the first liquid exchange assembly; a filtration membrane is built in the filter in a folded or stacked manner; and a gas permeation rate of the filter is 5 L/min-30 L/min, and an area of the filtration membrane is greater than 2,700 mm² wherein the method comprises:
S1: culture medium addition: adding a culture medium to the closed chamber of the reactor housing through the liquid communicating pipe;
S2: cell inoculation: adding a target cell to the closed chamber of the reactor housing through the liquid communicating pipe;
S3: cell culture: keeping the gas exchange assembly unobstructed during an entire process of cell culture to ensure a supply of a gas required for cell growth, and carrying out the cell culture in an incubator; and taking, during the culture process, a sample through the liquid communicating pipe to monitor a change in a nutrient in the culture medium, and adding a missing nutrient;
S4: culture medium discharge: communicating the liquid communicating pipe with a waste liquid flask, and discharging a waste culture medium from the closed chamber through a peristaltic pump; and allowing, during a discharge process, external air to enter the closed chamber after the external air passes through the filter to filter out a contaminant, thereby ensuring pressure balance inside the highly-breathable closed cell culture device; and
S5: cell collection: collecting cells through the second liquid exchange assembly.

11. A cell culture method, implemented by a highly-breathable closed cell culture device comprising:
a base;
a reactor housing, located above the base and connected to the base to form a closed chamber:
a gas exchange assembly, located above the base and communicated with the closed chamber;
a first liquid exchange assembly, located above the reactor housing and connected to the reactor housing; and
a second liquid exchange assembly, located on a side wall of the reactor housing and communicated with the closed chamber;
wherein, a filter is provided in the first liquid exchange assembly; a filtration membrane is built in the filter in a folded or stacked manner; and a gas permeation rate of the filter is 5 L/min-30 L/min, and an area of the filtration membrane is greater than 2.700 mm², and wherein a partition assembly, located inside the reactor housing and connected to an inner side wall of the reactor housing to divide the closed chamber into a first chamber and a second chamber, wherein the first chamber is located above the second chamber; and a volume ratio between the first chamber and the second chamber is 9:1,
wherein the method comprises:
S1: cell inoculation: diluting required cells with a culture medium that accounts for 10% of a volume of the closed chamber, and adding the cells to the second chamber of the reactor housing through the second liquid exchange assembly;
S2: culture medium addition: adding the culture medium to the first chamber through the liquid communicating pipe, wherein the added culture medium accounts for 90% of the volume of the closed chamber;
S3: cell culture: keeping the first breathable membrane and the gas exchange assembly unobstructed during an entire process of cell culture to ensure a supply of a gas required for cell growth, and carrying out the cell culture in an incubator;
S4: culture medium replacement: pumping out, in case of a need to replace the culture medium, a waste culture medium through the liquid communicating pipe; allowing, during a pumping process, external air to enter the closed chamber after the external air passes through the filter to filter out a contaminant, so as to ensure pressure balance inside the highly-breathable closed cell culture device; and feeding, after the pumping process is completed, a fresh culture medium through the liquid communicating pipe for cell growth; and
S5: concentrated cell collection: repeating the step S4 for multiple times to obtain multiple batches of mixed cultured cells, cell secretion product and culture medium in the second chamber, to obtain concentrated cells; communicating the first liquid exchange assembly with a waste liquid flask, and discharging the waste culture medium from the first chamber; mixing the cultured cells, the cell secretion product and the culture medium in the second chamber, and allowing the cultured cells and the cell secretion product to suspend in the culture medium; and opening the second liquid exchange assembly, and transferring and collecting the cultured cells, the cell secretion product and the culture medium through the second liquid exchange assembly.
